(19) United States
(12) Patent Application Publication
(10) Pub. No.: US 2016/0015841 A1
(43) Pub. Date: Jan. 21, 2016

(54) CONDENSATION ASSEMBLY AND METHOD

(71) Applicant: SCICAN LTD., Toronto (CA)

(72) Inventor: Stelian-Gabriel GAL, North York (CA)

(21) Appl. No.: 14/773,460

(22) PCT Filed: Mar. 11, 2014
(86) PCT No.: PCT/CA2014/050211

§ 371 Date: Sep. 8, 2015

Related U.S. Application Data
(60) Provisional application No. 61/776,369, filed on Mar. 11, 2013.

(51) Int. Cl. A61L 2/04; F28B 9/08; B08B 3/00

(52) U.S. Cl. CPC A61L 2/04; B08B 3/00; F28B 9/08

(57) ABSTRACT

A condenser assembly for a chamber of a sterilization system is provided which includes a cooling apparatus operable within the chamber and external means for receiving exhaust air and vapors from the chamber and for separating vapors into water and air. The external means may include an air-water separator or a condenser. The cooling apparatus is operable during at least one phase or cycle of a sterilization process to reduce the temperature inside the chamber and increase the condensation of vapors inside the chamber. A method of treating articles in a sterilization system with the condenser assembly includes disinfecting the articles in a chamber and, after disinfecting, recondensing vapor within the chamber using the cooling apparatus.

### RELATED APPLICATIONS

The present application claims priority from U.S. provisional application No. 61/776,369 filed Mar. 11, 2013, the contents of which are hereby incorporated by reference.

### FIELD OF THE INVENTION

The present invention relates to sterilization systems for medical or dental instruments and to methods and assemblies for condensing and exhausting vapors from the system.

### BACKGROUND

Sterilization systems are complex medical systems used to clean and disinfect medical instruments. Many support double functions of first, a thorough cleaning cycle, followed by a heat disinfection cycle where water temperature is elevated almost to boiling point. A drying cycle completes the sterilization process.

During the disinfection cycle, temperatures inside the chamber of the system must reach high levels sufficient to guarantee that the deactivation and/or killing of pathogenic microorganisms is fully accomplished. Due to the high temperature, close to the water boiling temperature, there is a considerable amount of vapors generated which will not be evacuated from the chamber at the same time as the hot water. The trapped vapors will be pushed out through the exhaust pipes of the chamber to the nearby surrounding environment once the drying cycle starts.

Evacuating or exhausting hot wet air from a sterilization system to the surrounding environment is inconvenient and creates a hazard for health and safety due to the high temperature vapors. As well, moisture will condensate on any colder surface and water droplets will accumulate locally damaging the cabinetry and/or creating a hazard for electrical safety. Evacuating or exhausting hot wet air from the system also increases the relative humidity of the surrounding environment which may make the air unhealthy for breathing.

### SUMMARY

A condenser assembly for a chamber of a sterilization system is provided which includes a cooling apparatus operable within the chamber and external means for receiving exhaust air and vapors from the chamber and for separating vapors into water and air. In one embodiment the external means comprises an air-water separator. The cooling apparatus is operable during at least one phase or cycle of a sterilization process to reduce the temperature inside the chamber and increase the condensation of vapors inside the chamber. The cooling apparatus may comprise a length pipe having an inlet and outlet from the chamber and configured in a pattern to exchange heat within the chamber.

According to an embodiment of the present there is provided a method of treating articles in a chamber in a sterilization system. The method includes disinfecting the articles in the chamber. After disinfecting, the method includes recondensing vapors within the chamber using a cooling apparatus. During recondensing, the method includes exhausting condensed vapors from the chamber to a reservoir, drain or other containment and/or disposal means and exhausting uncondensed vapors from the chamber to an external means for separating the uncondensed vapors into water and air. A cooling apparatus may be operable within the chamber to decrease the temperature of the chamber and increase the recondensation of vapors within the chamber. In one embodiment, disinfecting the articles includes raising an inside temperature of the chamber of the sterilization system to a disinfection level and exhausting air and vapors from the chamber to an external means for separating the vapors into water and air. In some embodiments, the method includes washing the articles prior to disinfecting. In some embodiments, the method includes drying the articles after disinfecting and recondensing vapors in the chamber.

### BRIEF DESCRIPTION OF THE DRAWINGS

These and other advantages of the present disclosure will become apparent upon reading the following detailed description and upon referring to the drawings in which:

FIG. 1 is a front perspective view of a condenser assembly in accordance with an embodiment of the present disclosure;

FIG. 2 is a rear perspective view of a condenser assembly in accordance with an embodiment of the present disclosure;

FIGS. 3A and 3B are perspective views of an air-water separator component of a condenser assembly in accordance with embodiments of the present disclosure;

FIG. 4 is a flowchart of a method in accordance with an embodiment of the present disclosure; and

FIGS. 5(a) to (d) are views of a condenser assembly in accordance with another embodiment of the present disclosure.

While the invention will be described in conjunction with the illustrated embodiments, it will be understood that it is not intended to limit the invention to such embodiments. On the contrary, it is intended to cover all alternatives, modifications and equivalents as may be included within the spirit and scope of the invention. The scope of the claims should not be limited by the preferred embodiments set forth in the examples, but should be given the broadest interpretation consistent with the specification as a whole.

### DETAILED DESCRIPTION OF THE PREFERRED EMBODIMENTS

In the following description, similar features in the drawings have been given identical reference numerals where appropriate. Terms such as “front” and “rear”, “top” and “bottom”, “first” and “second”, “right” and “left” may be used to identify opposing ends or different configurations of structures. Such terms are used for illustration purposes and are not intended to limit the present disclosure.

Methods and assemblies disclosed herein are provided for use in a sterilization system, such as sterilization system for medical and dental instruments including but not limited to a STATIM™ sterilization system. An embodiment of the present disclosure provides a method of treating articles in a sterilization system including condensing vapors inside a chamber of the sterilization system and a method of condensing vapors exiting the chamber. Also provided is a condenser assembly for use in a sterilization system. In some embodiments, a condenser assembly is provided for use in a sterilization system which performs both a cleaning or washing cycle and a disinfecting cycle. Such cycles may be followed by a drying cycle which completes the sterilization process. The use of the condenser assembly disclosed herein reduces the amount of vapors exhausted from the chamber of a sterilization system during a recondensation cycle after a disinfection cycle is complete. The use of the condenser assembly disclosed herein also reduces the amount of vapor trapped inside a chamber of the sterilization system after water has been drained, thus reducing the amount of gaseous water which eventually will be retained inside the chamber.

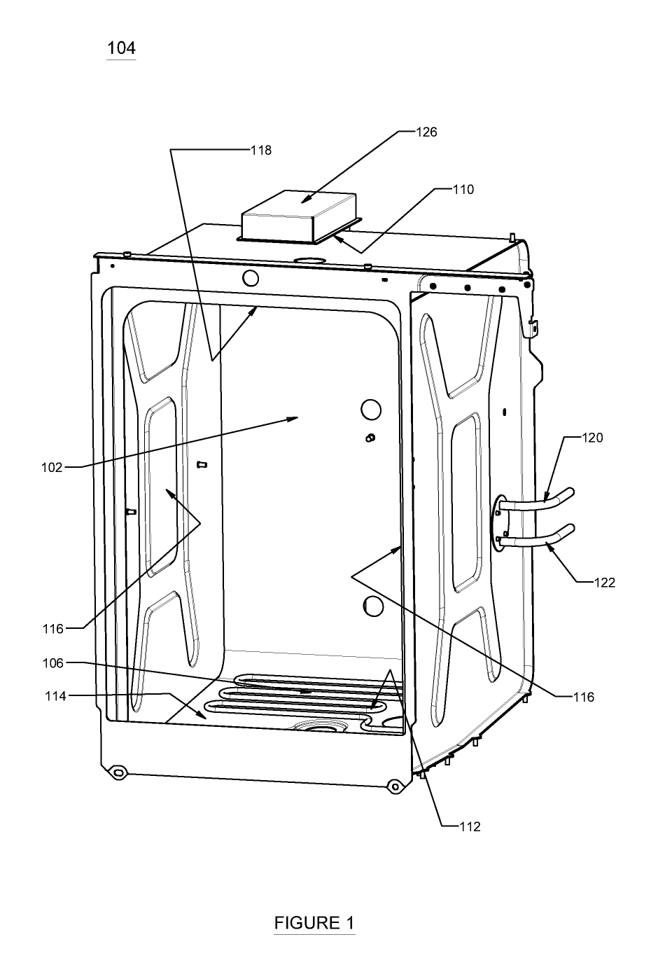

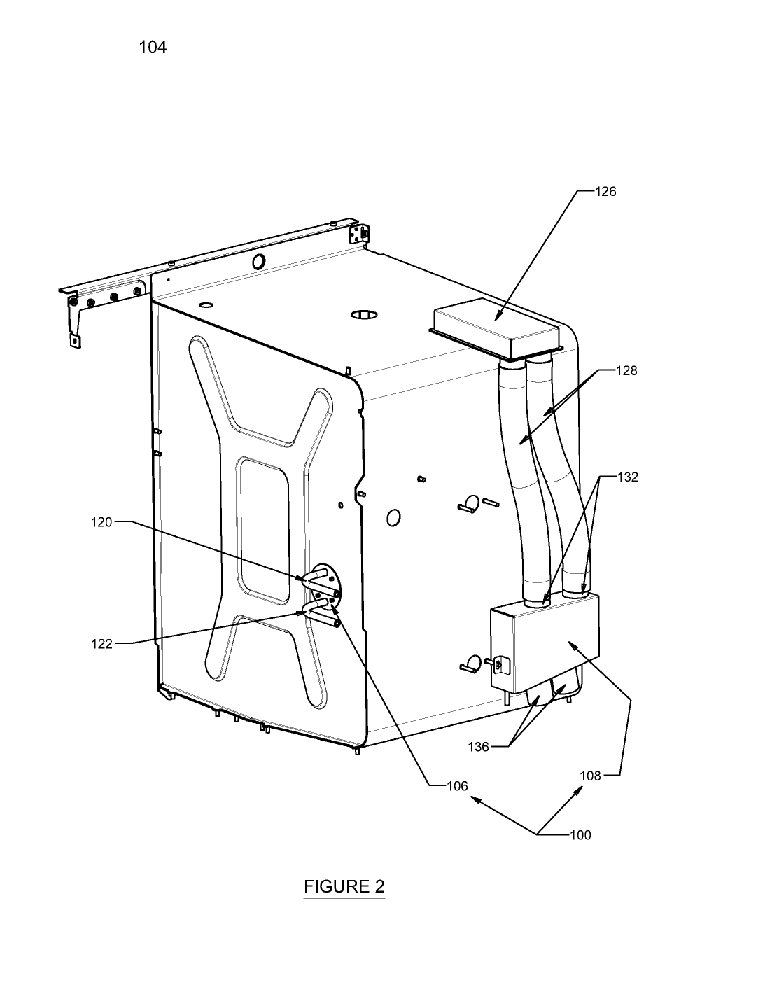

FIGS. 1 and 2 illustrate a condenser assembly 100 according to one embodiment of the present disclosure in conjunction with a chamber 102 of a sterilization system 104 (shown in a cut-away view). Other components of the sterilization system 104 which include but are not limited to generation means for steam or superheated means, distribution means, water reservoirs, pumps, valves, drains and controls are not shown. The chamber 102 also may include other components (not shown), including but not limited to a door, thermal insulation, and a drain system.

In one embodiment, the condenser assembly 100 includes a cooling apparatus 106 which is located within the chamber 102 and external means for separating air and water 108 which is located outside the chamber 102. The external means for separating air and water 108 is connected to an exhaust outlet 110 of the chamber 102. The cooling apparatus 106 is operable to reduce the temperature in the chamber 102 during a recondensation cycle, as described below, thus diverting or reducing water vapors which exit through the exhaust outlet 110 of the chamber 102 and which is condensed in order to reduce or prevent exhausting the vapors to the sterilization system environment.

In one embodiment, the cooling apparatus 106 comprises a pipe 112 or a stainless steel pipe. The pipe 112 may be arranged in the chamber 102 in a coiled, “zig-zag” or other pattern in order to increase the surface area of the pipe 112 which is exposed within the chamber 102. The length, size and pattern of pipe 112 in the cooling apparatus 106 may be varied to change the heat exchange efficiency of the apparatus 106. The cooling apparatus 106 may be located within the chamber 102, such as resting on or adjacent to a floor 114 of the chamber 102 as shown in FIG. 1. When adjacent the floor 114 of the chamber 102, cooling efficiency may be increased as the cooling apparatus 106 may be fully or partially immersed in the water to be cooled. In other embodiments, the cooling apparatus 106 is provided adjacent a wall 116 or roof 118 of the chamber 102. The cooling apparatus 106 includes one or more outlet ports to exterior of the chamber 102. In one embodiment, the cooling apparatus 106 includes an inlet port 120 and an outlet port 122. The inlet port 120 and outlet port 122 may be located through a side wall 116 of the chamber 102. As described below, during certain cycles of the sterilization system 104, a coolant is provided and flows through the cooling apparatus 106 via the inlet port 120 and outlet port 122. In one embodiment, the coolant comprises water. The coolant may be controlled and driven through the cooling apparatus 106 by pumps and controls, including computer controls, (not shown) of the sterilization system 104.

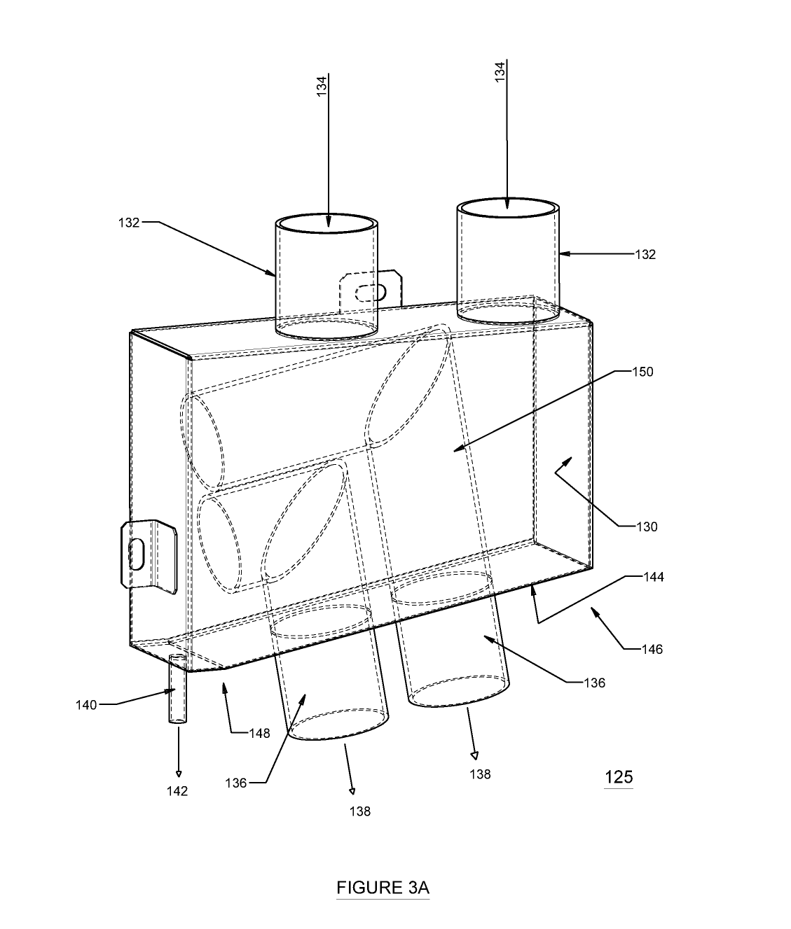

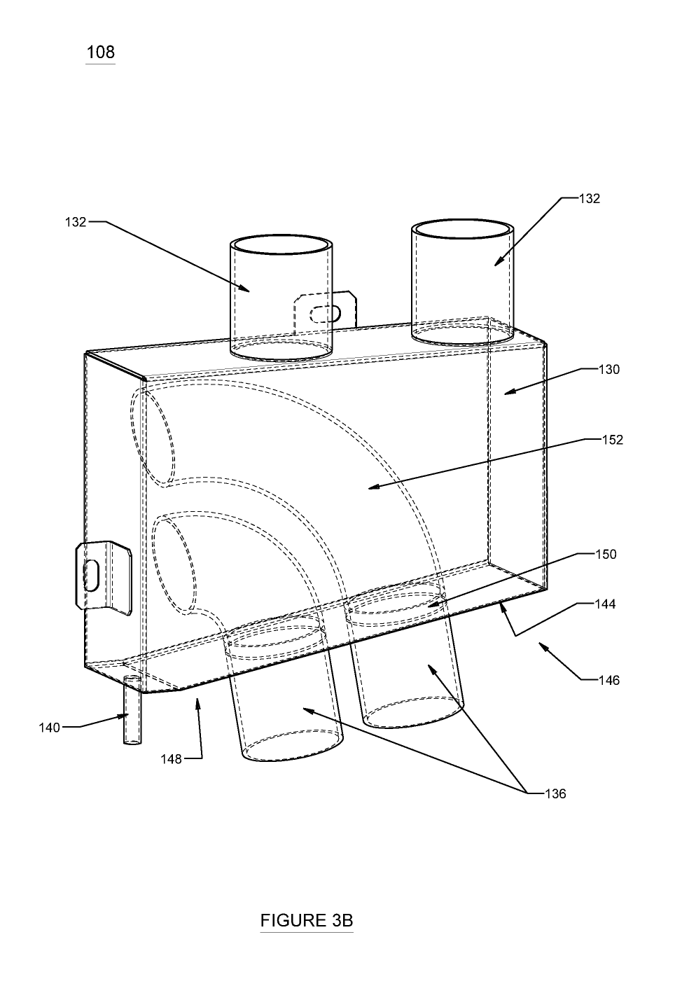

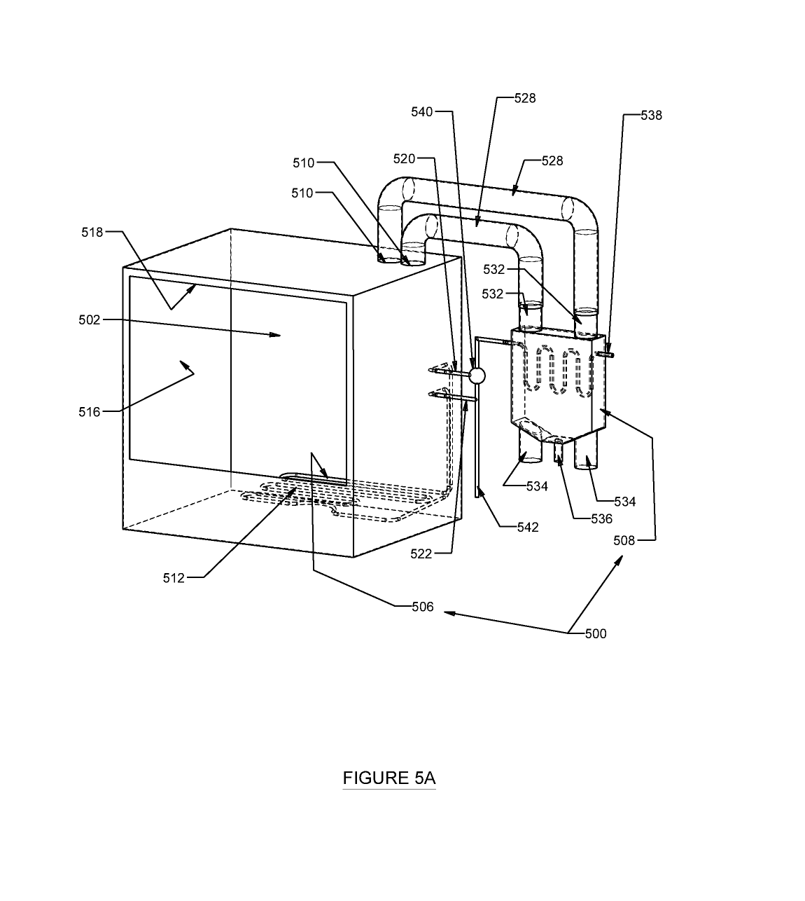

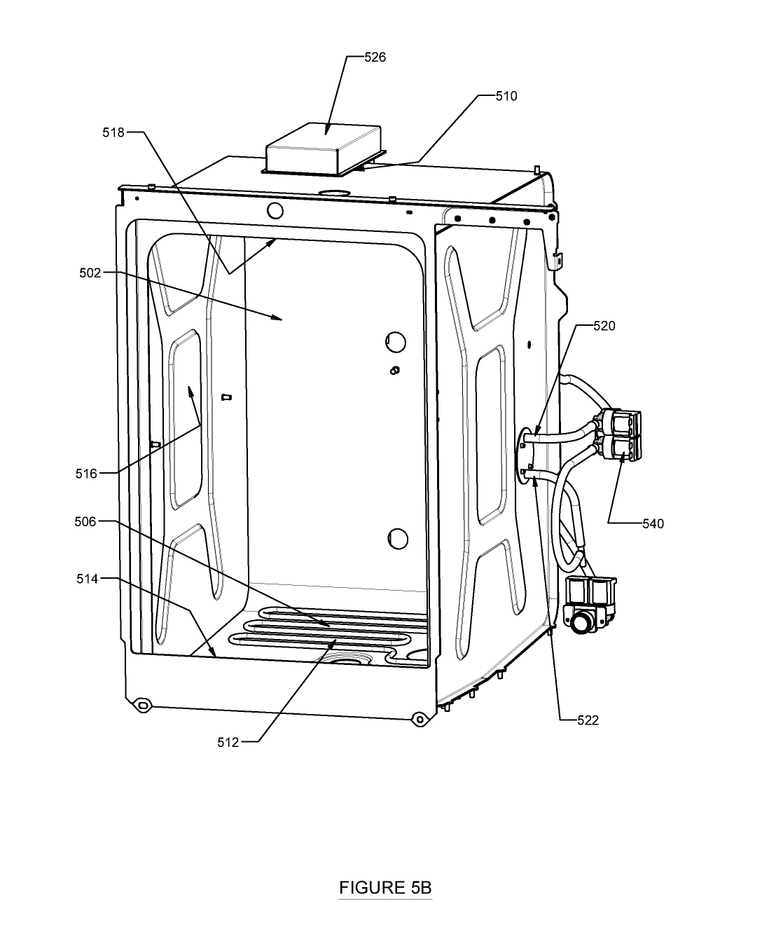

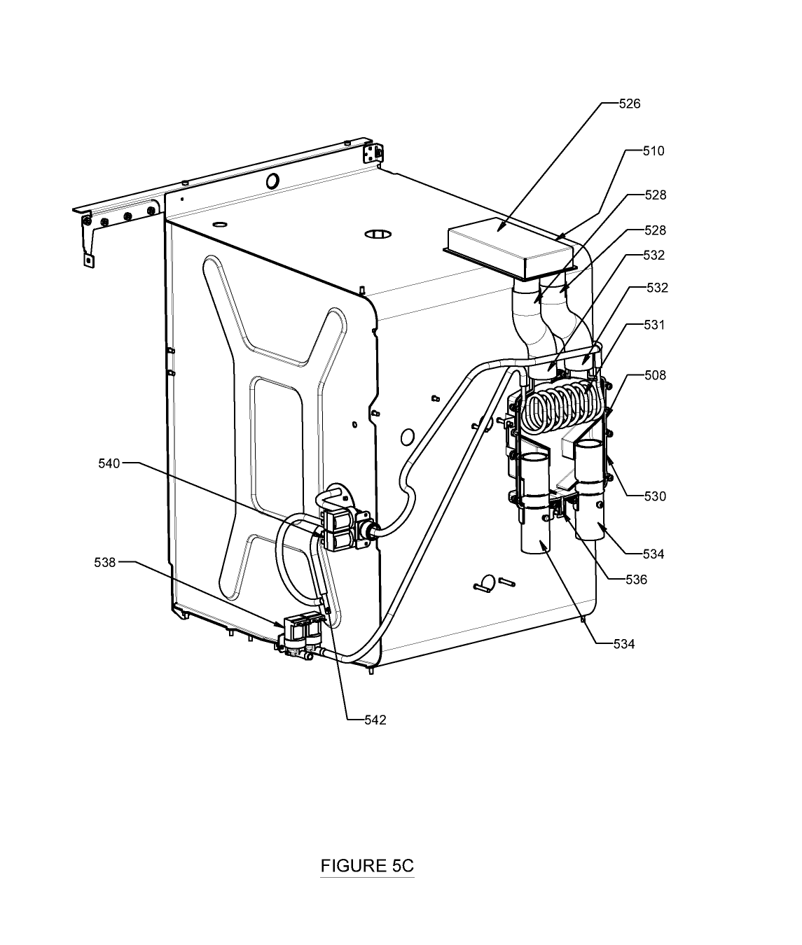

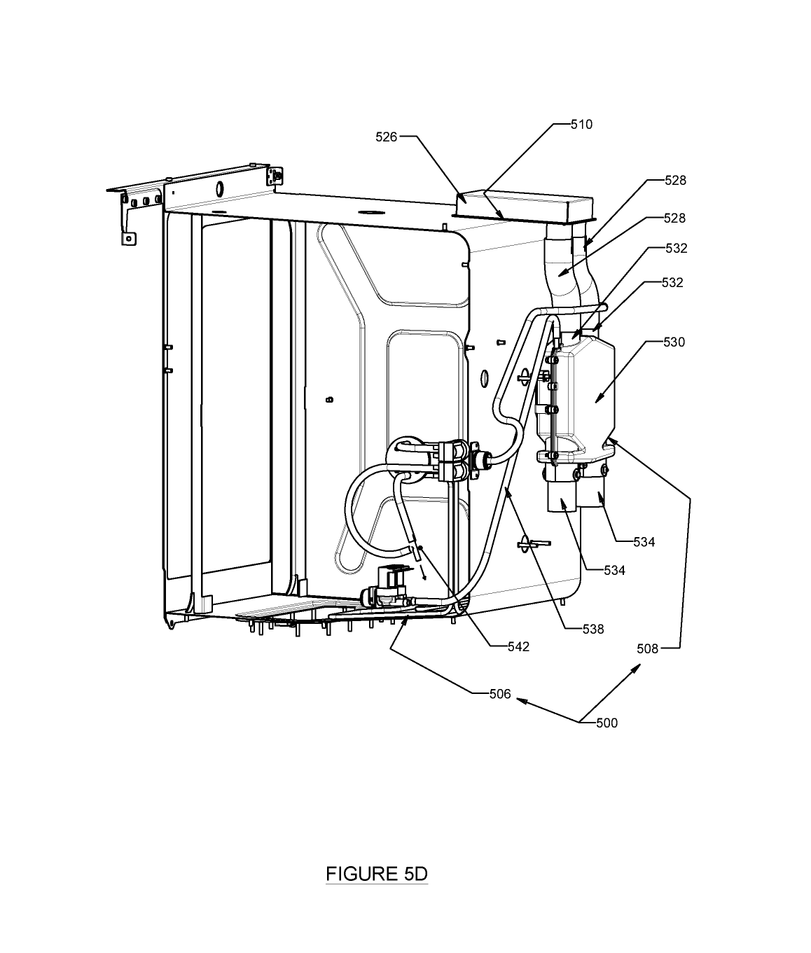

In one embodiment, the external means for separating air and water 108 separates or traps water droplets or condensate from the exhaust which exits the chamber 102. In one embodiment (see FIGS. 5(a) to (d)), the external means for separating air and water 108 comprises a condenser 508. In another embodiment, the external means for separating air and water 108 comprises an air-water separator 125 (see FIGS. 3A and 3B).

As shown in FIGS. 1 and 2, the external means for separating air and water 108 is in communication with the exhaust outlet 110 of the chamber 102. An air collector duct 126 may be provided adjacent the exhaust outlet 110 to direct air exhaust to the external means for separating air and water 108. In one embodiment, the air collector duct 126 is comprised of stainless steel. One or more air exhaust hoses 128 may be used to connect the air collector duct 126 to the external means for separating air and water 108.

FIGS. 3A and 3B show enlarged views of an air-water separator 125 according to embodiments of the present disclosure. The air-water separator 125 defines a housing 130 for receiving and separating or trapping water from the exhaust air and vapors in order to limit the discharge of vapors into the surrounding environment. In one embodiment, air-water separator 125 is comprised of stainless steel. The air-water separator 125 includes one or more inlet ports 132 for receiving exhaust air and vapors (“wet air” indicated by arrows 134) from the chamber 100; one or more outlet ports 136 for discharging air or primarily “dry air” (indicated by arrows 138) to the surrounding environment and one or more outlet ports 140 for discharging condensed vapors (indicated by arrows 142) to a reservoir, drain or other containment and/or disposal means (not shown).

The air-water separator 125 is configured to create a circulation or spin of air and vapors as they are received from the chamber 102 of the sterilization system 104. This circulation creates a centrifugal force which pushes condensated water droplets towards walls of the housing 130. Condensated water droplets collide with each other and form larger droplets that adhere to inner surfaces of the walls of the housing 130. As these droplets increase in size and weight, they drop downwardly towards a bottom or floor 144 of the housing 130. In one embodiment, the floor 144 of the housing 130 is inclined with a first end 146 being higher than the second end 148. The one or more outlet ports 140 for discharging condensed vapors 142 are located in the second end 148 of the floor to receive and discharge condensed vapors 142. In one embodiment, the first end 146 of the floor 144 is adjacent the one or more inlet ports 132 for receiving exhaust air and vapors.

In one embodiment, the one or more outlet ports 136 for discharging air or primarily “dry air” to the surrounding environment define one or more channels 150 which extend within the housing 130. The channels 150 are configured to accommodate and promote the circulation of air and vapors within the housing 130 and to delay the travel of air between the one or more inlet ports 132 and the one or more outlet ports 136 for discharging air 138 from the condenser 125. In one embodiment, the one or more channels 150 include an angled portion. In another embodiment, as illustrated by the lined portions of FIG. 3B, the one or more channels 150 may include a curved portion 152 extending within the housing 130 and away from the one or more inlet ports 132. Other embodiments and configurations of the one or more channels 150 may be provided depending on manufacturing constraints and performance criteria.

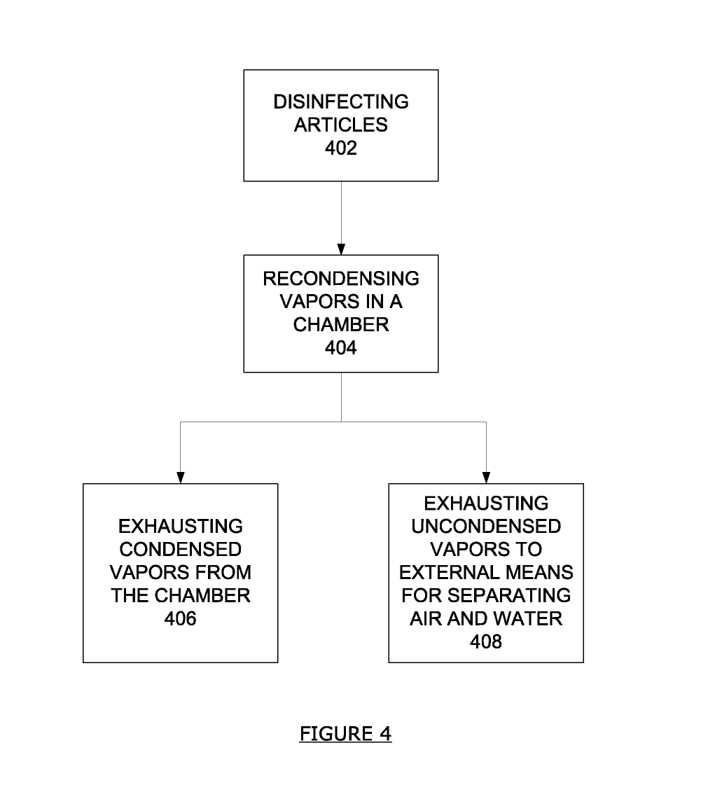

FIG. 4 shows a method 400 of treating articles in a chamber 102 in a sterilization system 104 according to the present disclosure. In one embodiment, the method includes disinfecting the articles 402 in the chamber 102. After disinfecting, the method includes recondensing vapors 404 within the chamber 102 using a cooling apparatus 106. During recondensing, the method 400 includes exhausting condensed vapors 406 from the chamber 102 and exhausting uncondensed vapors 408 from the chamber 102 to an external means for separating the vapors into water and air 108. In one embodiment, disinfecting the articles 402 includes raising an inside temperature of the chamber 102 of the sterilization system 104 to a disinfection level and exhausting air and vapors from the chamber 102 to an external means for separating the vapors into water and air 108. In some embodiments, the method 400 includes washing the articles prior to disinfecting 402. In some embodiments, the method includes drying the articles after disinfecting 402 and recondensing vapors 404 in the chamber 102.

It will be appreciated that the condenser assembly 100 and methods 400 disclosed herein may be used in one phase or cycle, one or more phases or cycles of a sterilization process or in all phases or cycles of the process. In one embodiment, the condenser assembly 100 and methods 400 of the present disclosure are operable to condense and exhaust vapors during two condensing phases of a sterilization process.

One phase occurs during a disinfection cycle of the sterilization process which includes increasing the temperature inside the chamber 102 to a first temperature sufficient to deactivate and/or kill pathogenic microorganisms. In one embodiment, the inside temperature of the chamber 102 is increased to a first temperature of 93 degrees Celsius. The increasing temperature inside the chamber 102 creates a positive relative pressure that pushes generated steam in the chamber 102 to an exhaust outlet 110 of the chamber 102, through one or more air exhaust hoses 128, to the external means for separating the vapors into water and air 108. Due to the temperature difference between the external means for separating the vapors into water and air 108 and the chamber 102, most of the vapors condensate inside an enclosure or housing of the external means for separating the vapors into water and air 108. In one embodiment, the external means for separating the vapors into water and air 108 is closer to ambient temperature and thus improves conditions for the condensation of vapors. During this phase, the cooling apparatus 106 of the present disclosure is passive. Moisture in the exhaust from the chamber 102 is fully condensated in the external means for separating the vapors into water and air 108 and substantially no water in a liquid or gaseous phase reaches the one or more outlet ports 136 for discharging air or primarily “dry air” to the surrounding environment.

Another phase starts once the disinfection cycle is complete and ends once the inside temperature decreases from the first temperature to a second temperature. This phase is referred to herein as a recondensation phase. In one embodiment, the second temperature is approximately 70 degrees C.

During this recondensation phase, the cooling apparatus 106 is operable to reduce the temperature of the chamber 102. In one embodiment, a coolant such as cooling water is distributed through the cooling apparatus 106 in the chamber 102. In one embodiment as described above, the cooling apparatus 106 comprises a pipe 112 and the heat exchange efficiency of the apparatus 106 varies with the size, length, material and configuration of the pipe 112 within the chamber 102. In one embodiment, the cooling rate or speed of temperature decrease varies between 4.4 to 6 degrees C. per minute. In one embodiment, the rate of flow of cooling water through the cooling apparatus 106 varies between 1.1 to 1.5 liters/minute. In one embodiment, the time needed to cool the chamber 102 and its load to the second temperature is about 3.8 to 5.2 minutes using approximately 4.2 to 7.8 liters of cooling water. Cooling rates vary with the load of articles in the chamber 102 and lower rates may be obtained if the chamber 102 includes a full load of articles such as medical or dental instruments or cassettes containing such instruments. As noted above, the control and temperature of coolant through the cooling apparatus 106 may be controlled or programmed within or by the controls (not shown) of the sterilization system 104.

During this recondensation phase, most vapors recondensate inside the chamber 102 of the sterilization system 104 as its temperature decreases. The recondensated vapors exit or are discharged from the chamber 102 through a drain system (not shown) to a reservoir, drain or other containment and/or disposal means. In one embodiment, the recondensated vapors exit or are discharged from the chamber 102 in the same time and through the same drain system as the discharge of water used for washing articles in the chamber 102. Uncondensed vapors which exit from the chamber 102 through the exhaust outlet 110 are directed to the external means for separating the vapors into water and air 108 and are substantially fully condensated before air exits the condenser assembly 100 to the surrounding environment.

Once the chamber 102 reaches the second temperature, a next phase starts, referred to herein as a drying phase. During this phase, the cooling apparatus 106 of the present disclosure is passive. Any remaining vapors exiting the chamber 102 through the exhaust outlet 110 are recondensated by the external means for separating the vapors into water and air 108.

In one embodiment of the present disclosure, with the use of the cooling apparatus 106 in the chamber 102, as described above, an external condenser also using coolant or cooling water to recondensate the exhaust air and vapors from the chamber is not required. The external means for separating the vapors into water and air 108 may comprise an air-water separator which does not require additional coolant or cooling water.

FIGS. 5(a) to (d) illustrate a condenser assembly 500 according to one alternative embodiment of the present disclosure in conjunction with a chamber 502 of a sterilization system 504 (shown in a cut-away view). Other components of the sterilization system 504 which include but are not limited to generation means for steam or superheated means, distribution means, water reservoirs, pumps, valves, drains and controls are not shown. The chamber 502 also may include other components (not shown), including but not limited to a door, thermal insulation, and a drain system.

In one embodiment, the condenser assembly 500 includes a cooling apparatus 506 which is located within the chamber 502 and external means for separating air and water comprising a condenser 508 which is located outside the chamber 502. The condenser 508 is connected to an exhaust outlet 510 of the chamber 502. The cooling apparatus 506 is operable to reduce the temperature in the chamber 502 during a recondensation cycle, as described below, thus diverting or reducing water vapors which exit through the exhaust outlet 510 of the chamber 502 and which is condensed in order to reduce or prevent exhausting the vapors to the sterilization system environment.

In one embodiment, the cooling apparatus 506 comprises a pipe or a stainless steel pipe 512. The pipe 512 may be arranged in the chamber 502 in a coiled, “zig-zag” or other pattern in order to increase the surface area of the pipe 512 which is exposed within the chamber 502. The length, size and pattern of pipe 512 in the cooling apparatus 506 may be varied to change the heat exchange efficiency of the apparatus 506. The cooling apparatus 506 may be located within the chamber 502, such resting on or adjacent to a floor 514 of the chamber 502 as shown in FIGS. 5(a) and 5(b). When adjacent the floor 514 of the chamber 502, cooling efficiency may be increased as the cooling apparatus 506 may be immersed in the water to be cooled. In other embodiments, the cooling apparatus 506 is provided adjacent a wall 516 or roof 518 of the chamber 502. The cooling apparatus 506 includes one or more outlet ports to exterior of the chamber 502. In one embodiment, the cooling apparatus 506 includes an inlet port 520 and an outlet port 522. The inlet port 520 and outlet port 522 may be located through a side wall 516 of the chamber 502. As described below, during certain cycles of the sterilization system 504, a coolant is provided and flows through the cooling apparatus 506 via the inlet port 520 and outlet port 522. In one embodiment, the coolant comprises water. The coolant may be controlled and driven through the cooling apparatus 506 by pumps and controls, including computer controls, (not shown) of the sterilization system 504.

As shown in FIGS. 5(a), 5(c) and 5(d), the condenser 508 is in communication with the exhaust outlet 510 of the chamber 502. An air collector duct 526 may be provided adjacent the exhaust outlet 510 to direct air exhaust to the condenser 508. In one embodiment, the air collector duct 526 is comprised of stainless steel. One or more air exhaust hoses 528 may be used to connect the air collector duct 526 to the condenser 508.

In one embodiment, the condenser 508 includes a condenser body 530 which houses a condenser coil 531 which are operable to receive and separate water from the exhaust air and vapors in order to limit the discharge of vapors into the surrounding environment. The condenser 508 includes one or more inlet ports 532 for receiving exhaust air and vapors (“wet air”) from the chamber 502; one or more outlet ports 534 for discharging air or primarily “dry air” to the surrounding environment and one or more outlet ports 536 for discharging condensed vapors to a reservoir, drain or other containment and/or disposal means (not shown).

In one embodiment, coolant or cooling water is provided to the condenser 508 from a cooling water inlet 538 and then to the cooling apparatus 506 in the chamber 502. A 3-way directional valve 540 may be provided and used to control the flow of coolant. A cooling water outlet 542 connected to the 3-way directional valve 540 and the outlet port 522 may be used to discharge cooling water.

During a disinfecting phase, as described above, coolant is provided to the condenser 508 but not to the cooling apparatus 506. The condenser 508 operates to remove water from the air and vapors exiting the exhaust outlet 510 of the chamber 502.

During a recondensation phase, coolant flows through the condenser 508 and the 3-way directional valve 540 is configured to provide coolant to the cooling apparatus 506. Coolant exits the cooling apparatus 506 and the chamber 502 to a reservoir, drain or other containment and/or disposal means. During the recondensation phase, most of the vapors recondensate in the chamber 502 as its temperature decreases and the recondensated vapors exit or are discharged from the chamber 502 through a condensate outlet (not shown) to a reservoir, drain or other containment and/or disposal means. Uncondensed vapors which exit from the chamber 502 through the exhaust outlet 510 are directed to the condenser 508 and are substantially fully condensated before air exits the condenser assembly 500 to the surrounding environment.

During all or a portion of a drying phase, coolant is provided to the condenser 508 and not to the cooling apparatus 506. The 3-way directional valve 540 is configured to block the path of coolant from the cooling apparatus 506 and divert coolant to the reservoir or drain. Any remaining vapors in the chamber 502 are pushed through the exhaust outlet 510 of the chamber 502 and condensated inside the condenser 508.

Thus, it is apparent that there has been provided in accordance with the invention methods and assemblies for the condensation and removal of vapors from a sterilization system that fully satisfy the objects, aims and advantages set forth above. While the invention has been described in conjunction with illustrated embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

1. A method of treating articles in a chamber in a sterilization system comprising:
disinfecting the articles;
after disinfecting, recondensing vapors within the chamber using a cooling apparatus;
and during recondensing,
exhausting condensed vapors from the chamber; and
exhausting uncondensed vapors from the chamber to an external means for separating the uncondensed vapors into water and air, the uncondensed vapors being exhausted via an exhaust outlet of the chamber to an air collector duct adjacent the exhaust outlet, the air collector duct directing the vapors from the chamber to the external means for separating the uncondensed vapors.

2. The method of claim 1 wherein disinfecting the articles comprises raising an inside temperature of the chamber of the sterilization system to a disinfection level and exhausting air and vapors from the chamber to the external means for separating the uncondensed vapors into water and air.

3. The method of claim 1 further comprising drying the articles.

4. The method of claim 1 further comprising, prior to disinfecting the articles, washing the articles.

5. The method of claim 1 wherein the external means for separating the uncondensed vapors into water and air comprises an air-water separator.

6. The method of claim 1 wherein the external means for separating the uncondensed vapors into water and air comprises a condenser.

7. The method of claim 1 wherein recondensing vapor within the chamber using a cooling apparatus comprises distributing coolant through the cooling apparatus.

8. A condenser assembly for a chamber of a sterilization system comprising:
a cooling apparatus operable within the chamber;
external means for receiving exhaust air and vapors from the chamber and for separating vapors into water and air; and
an air collector duct adjacent an exhaust outlet of the chamber to direct exhaust air and vapors from the chamber to the external means for separating vapors into water and air.

9. The condenser assembly of claim 8 further comprising at least one exhaust hose connecting the air collector duct to the external means for separating vapors into water and air.

10. The condenser assembly of claim 8 wherein the cooling apparatus comprises a pipe configured in a pattern to increase heat exchange efficiency.

11. The condenser assembly of claim 10 wherein the cooling apparatus comprises a stainless steel pipe.

12. The condenser assembly of claim 8 wherein the means for separating vapors into water and air comprises a condenser.

13. The condenser assembly of claim 8 wherein the means for separating vapors into water and air comprises an air-water separator.

14. The condenser assembly of claim 13 wherein the air-water separator comprises a housing configured to receive and circulate exhaust air and vapors.
